(19) United States
(12) Patent Application Publication
(10) Pub. No.: US 2010/0125189 A1
(43) Pub. Date: May 20, 2010

(54) SYSTEMS AND METHODS FOR CATHETER STABILITY

(75) Inventors: Jason Jacobson, Chicago, IL (US); Jason Rubenstein, Naperville, IL (US); Michael Kim, Wilmette, IL (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/620,178

(22) Filed: Nov. 17, 2009

Related U.S. Application Data
(60) Provisional application No. 61/115,342, filed on Nov. 17, 2008.

(51) Int. Cl. A61B 5/042

(52) U.S. Cl. 600/381

(57) ABSTRACT

The present invention provides systems and methods for stabilizing catheters, such as decapolar coronary sinus catheters in veins. In particular, the present invention provides fixation elements for improved tissue contact and stability.

The present application claims priority to U.S. Provisional application 61/115,342, filed Nov. 17, 2008, which is herein incorporated by reference in its entirety.

### FIELD OF THE INVENTION

The present invention provides systems and methods for stabilizing catheters, such as decapolar coronary sinus catheters in veins. In particular, the present invention provides fixation elements for improved tissue contact and stability.

### BACKGROUND OF THE INVENTION

Advanced arrhythmia ablation techniques in cardiac electrophysiology have become increasing reliant on electroanatomic mapping and navigation. One of the major products is the Ensite NAVX, which is an impedance-based system that is most optimally operated using one of the intracardiac catheters as a navigation reference. A major drawback of this strategy is that any shift in the location of the reference catheter will disrupt the accuracy of the map, which is a major issue for both ablation effectiveness and patient safety. Often, maps must be recreated several times during a procedure due to reference catheter movement, which lengthens the procedure duration significantly. The most commonly used catheter used for reference is the coronary sinus catheter, which is a standard steerable decapolar catheter. The position of these catheters is maintained passively, and remaining in place only by mild friction between the catheter shaft and the vein.

### SUMMARY

In some embodiments, the present invention provides a system comprising a steerable catheter (e.g., a steerable decapolar coronary sinus catheter), wherein said catheter further comprises active fixation elements. In some embodiments, the catheter comprises two or more (e.g., 3, 5, 6, 7, 8, 9, 10, 11, 12, etc.) active fixation elements. In some embodiments, the fixation elements are extendable and/or retractable. In some embodiments, the fixation elements extend perpendicularly to the longitudinal axis of said catheter. In some embodiments, the fixation elements extend at approximately 0°, 90°, 180° and 270° around said longitudinal axis of said catheter. In some embodiments, the fixation elements are extendable and retractable in groups comprising one or more fixation elements at the distal end of said catheter and one or more fixation elements at the proximal end of said catheter.

In some embodiments, the present invention provides a method of stabilizing a catheter (e.g., decapolar coronary sinus catheter) to a tissue surface (e.g., within a vein): comprising positioning said catheter in the desired position with respect to tissue, and extending one or more fixation elements. In some embodiments, the fixation elements are configured to contact a coronary sinus wall. In some embodiments, contact between the said fixation elements and said coronary sinus wall stabilizes the position of said catheter. In some embodiments, the fixation elements extend perpendicularly to the longitudinal axis of said catheter. In some embodiments, the fixation elements comprises one or more fixation elements at the distal end of the catheter and one or more fixation elements at the proximal end of said catheter.

In some embodiments, the present invention provides a method of selectively placing a decapolar coronary sinus catheter against a desired wall of the coronary sinus, wherein one or more fixation elements are extended on sides of the catheter opposite said desired wall of the coronary sinus. In some embodiments, contact between the said fixation elements and said coronary sinus wall stabilizes the position of said catheter against opposite said desired wall of the coronary sinus. In some embodiments, the fixation elements extend perpendicularly to the longitudinal axis of said catheter.

### BRIEF DESCRIPTION OF THE DRAWINGS

The foregoing summary and detailed description is better understood when read in conjunction with the accompanying drawings which are included by way of example and not by way of limitation.

FIG. 1 shows a schematic of a side view of an exemplary steerable coronary sinus electrophysiology catheter with active fixation elements.

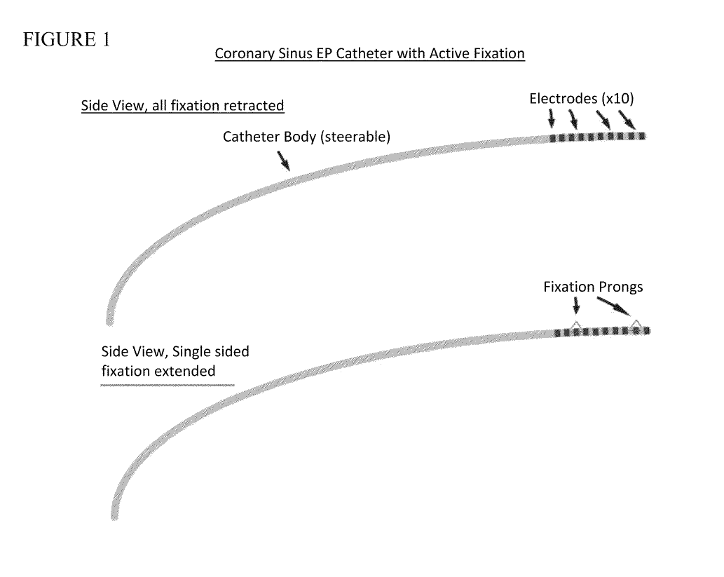

FIG. 2 shows a schematic of a head-on view of an exemplary steerable coronary sinus electrophysiology catheter with active fixation elements.

### DETAILED DESCRIPTION OF EMBODIMENTS

Most commonly, coronary sinus catheters are used passively, and remain in place only by mild friction between the catheter shaft and the vein. The catheter design of embodiments of the present invention incorporates, for example, a steerable decapolar coronary sinus catheter with active fixation elements for improved stability for use as a reference catheter. In addition, the fixation elements can be used for improved coronary sinus contact for better electrograms, or for selectively choosing to contact one wall of the coronary sinus. In some embodiments, fixation elements are provided in the form of fixation prongs. It should be understood that the catheters of the invention may find use in a wide variety of settings where more stable positioning is desired or needed.

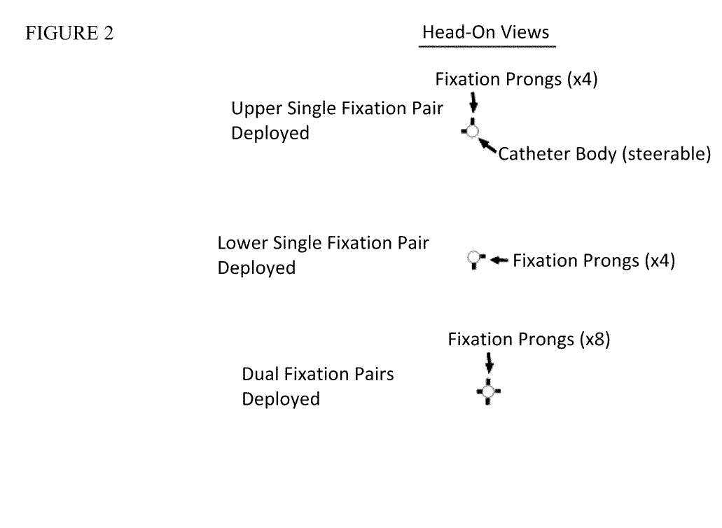

In some embodiments, the present invention provides a total of eight fixation elements which can be individually extended or retracted in two sets of 4, or two basic fixation controls. In some embodiments, each control extends 2 fixation elements near the distal electrodes and 2 elements near the proximal electrodes. In some embodiments, these two fixation elements are orientated at 90 degree angles to one another; i.e. the first set consists of two elements at 0 and 90 degrees proximally and two elements at 0 and 90 degrees distally, and the second set consists of two elements at 180 and 270 degrees proximally and 180 and 270 degrees distally (SEE FIG. 2). In some embodiments, other arrangements and groupings of the fixation elements are contemplated. In some embodiments, a fixation control controls the fixation elements on one side of the catheter. In some embodiments, the catheter provides fixation elements on one, two, three, or four sides. In some embodiments, the catheter provides proximally or distally located fixation elements. In some embodiments, the fixation element controls are sliders located on the handle, near the standard deflection control. In some embodiments there are one or more fixation element controls (e.g. 1 control, 2 controls, 3 controls, 4 controls, 5 controls, 6 controls, etc,). In some embodiments, the fixation elements are part of the catheter outer coating which “bunches up” in the extended position. In some embodiments, all control sets are retracted, and the catheter is fully passive for placement or removal. In some embodiments, all control sets are extended, for maximal fixation security. In some embodiments, one or more control sets are extended and one or more control sets are retracted, to provide electrode contact with coronary sinus as well as catheter stability.

In some embodiments, the present invention provides a catheter having a mapping assembly at its distal end. In some embodiments, the catheter comprises an elongated catheter body having proximal and distal ends, a control handle at the proximal end of the catheter body, and a mapping assembly mounted at the distal end of the catheter body. In some embodiments, the catheter body includes an elongated proximal shaft at its proximal end and a shorter distal shaft at its distal end. In some embodiments, the proximal shaft comprises an elongated tubular construction having a single, axial or central lumen. In some embodiments, the proximal shaft is flexible, e.g., bendable, but substantially non-compressible along its length. The proximal shaft can be of any suitable construction and made of any suitable material. A suitable construction includes, but is not limited to, an outer wall made of polyurethane or PEBAX, the outer wall comprising an imbedded braided mesh of stainless steel or the like, as is generally known in the art, to increase torsional stiffness of the proximal shaft so that, when the control handle is rotated, the distal shaft will rotate in a corresponding manner. In some embodiments, torsional stiffness is achieved through other mechanisms known to those in the art. In some embodiments, the useful length of the catheter, i.e., that portion that can be inserted into the body excluding the mapping assembly can vary as desired. In some embodiments, the useful length ranges from about 50 cm to about 200 cm. The length of the distal shaft is generally a relatively small portion of the useful length, for example from about 2 cm to about 20 cm, or from about 4 cm to about 8 cm.

In some embodiments, the mapping assembly comprises a generally straight region. The straight region is mounted on the distal shaft, so that its axis is generally parallel to the axis of the distal shaft. In some embodiments, the mapping assembly is formed of a non-conductive cover, which is preferably generally tubular, but can have any cross-sectional shape as desired. The non-conductive cover can be pre-formed into the desired generally shape. In some embodiments, the mapping assembly comprises the active fixation elements of the present invention. In some embodiments the mapping assembly comprises electrodes configured to perform electroanatomic mapping and navigation. The number of electrodes on the assembly can vary as desired. In some embodiments, the number of electrodes ranges from about six to about twenty. In some embodiments, the number of electrodes ranges from about eight to about twelve. In some embodiments, the assembly carries ten electrodes. In some embodiments, the electrodes are approximately evenly spaced. In some embodiments, a distance of approximately 1-20 mm is provided between the centers of the electrodes.

In some embodiments, lead wires attached to the electrodes extend through the lumen of the distal shaft, through the catheter body, and terminate at the proximal end of the catheter. In some embodiments, the portion of the lead wires extending through the central lumen of the catheter is enclosed within a protective sheath, which can be made of any suitable material. In some embodiments, the protective sheath is anchored at its distal end to the proximal end of the distal shaft.

We claim:

1. A system comprising a steerable decapolar coronary sinus catheter, wherein said catheter comprises one or more active fixation elements.

2. The system of claim 1, wherein said catheter comprises one or more distally located electrodes.

3. The system of claim 1, wherein said catheter comprises 8 active fixation elements.

4. The system of claim 1, wherein said fixation elements are extendable and retractable.

5. The system of claim 1, wherein said fixation elements extend perpendicularly to the longitudinal axis of said catheter.

6. The system of claim 5, wherein said fixation elements extend at 0°, 90°, 180° and 270° around said longitudinal axis of said catheter.

7. The system of claim 1, wherein said fixation elements are extendable and retractable in groups comprising one or more fixation elements at the distal end of said catheter and one or more fixation elements at the proximal end of said catheter.

8. A method of stabilizing a decapolar coronary sinus catheter within a vein comprising positioning said catheter in the desired position within a vein, and extending one or more fixation elements.

9. The method of claim 8, wherein said fixation elements are configured to contact the coronary sinus wall.

10. The method of claim 8, wherein said contact between the said fixation elements and said coronary sinus wall stabilizes the position of said catheter.

11. The method of claim 8, wherein said fixation elements extend perpendicularly to the longitudinal axis of said catheter.

12. The method of claim 8, wherein each said group of said fixation elements comprises one or more fixation elements at the distal end of the catheter and one or more fixation elements at the proximal end of said catheter.

13. A method of selectively placing a decapolar coronary sinus catheter against a desired wall of the coronary sinus, wherein one or more fixation elements are extended on sides of the catheter opposite said desired wall of the coronary sinus.

14. The method of claim 13, wherein contact between the said fixation elements and said coronary sinus wall stabilizes the position of said catheter against the opposite wall of the coronary sinus.

15. The method of claim 13, wherein said fixation elements extend perpendicularly to the longitudinal axis of said catheter.
